(19) United States
(12) Patent Application Publication
(10) Pub. No.: US 2019/0192212 A1
(43) Pub. Date: Jun. 27, 2019

(54) CONDUCTIVE ADHESION PREVENTING FILM FOR MEDICAL USE AND MEDICAL DEVICE

(71) Applicant: OLYMPUS CORPORATION, Tokyo (JP)

(72) Inventors: Yu MURANO, Tokyo (JP); Takuya FUJIHARA, Tokyo (JP); Hiroaki KASAI, Tokyo (JP); Takeshi DEGUCHI, Tokyo (JP)

(73) Assignee: OLYMPUS CORPORATION, Tokyo (JP)

(21) Appl. No.: 16/290,163

(22) Filed: Mar. 1, 2019

Related U.S. Application Data
(63) Continuation of application No. PCT/JP2017/039579, filed on Nov. 1, 2017.

(30) Foreign Application Priority Data
Nov. 11, 2016 (JP) 2016-220718

(51) Int. Cl. A61B 18/14; A61L 31/10

(52) U.S. Cl. CPC A61B 18/14; A61B 2018/00125; A61L 31/10

(57) ABSTRACT

A conductive adhesion preventing film for medical use, includes: a nonconductive base material; and a linear conductor having a length of 10 μm or more and a diameter of more than 50 nm and contained in the conductive adhesion preventing film by an amount of 5% by mass or more and 40% by mass or less, wherein the conductive adhesion preventing film is formed on an electrode surface of a medical device performing at least one of incision, resection, coagulation, and ablation on living tissue by applying a high frequency voltage.

### CROSS REFERENCE TO RELATED APPLICATIONS

This application is a continuation application based on a PCT Patent Application No. PCT/JP2017/039579, filed on Nov. 1, 2017, whose priority is claimed on Japanese Patent Application No. 2016-220718, filed on Nov. 11, 2016. The contents of both the PCT Application and the Japanese Application are incorporated herein by reference.

### BACKGROUND

### Technical Field

The present invention relates to a conductive adhesion preventing film for medical use, and a medical device.

### Background Art

As a medical device, an apparatus for applying a high-frequency voltage to a living tissue is known. For example, a high-frequency treatment instrument, which is an example of such a medical device, incises, coagulates or cauterizes a living tissue by applying a high-frequency voltage to a living tissue.

In such a medical device, in order to satisfy the treatment function with respect to the living tissue, a portion of the surface in contact with the living tissue is required to have conductivity. However, because metals with good conductivity are easy to adhere to living tissues, when the surface in contact with living tissue is made of metal, the durability of the medical device tends to decrease.

For example, Japanese Unexamined Patent Application, First Publication No. 2006-68407 describes a technique for improving the adhesion preventing property to living tissue by preventing oxidation of the surface of the high frequency electrode. In this document, it is described that a film of an alloy of gold or white metal is formed on the surface of the high-frequency electrode in order to prevent oxidation of the surface of the high-frequency electrode.

In a technical field other than a medical device application, a technique for dispersing a conductor in an insulator to obtain conductivity is known.

For example, in Japanese Unexamined Patent Application, First Publication No. 2009-96110 in the technical field of an electrophotographic apparatus, it is described that conductive particles are contained in a rubber material in order to maintain electrical conductivity in a rubber material.

For example, in Japanese Unexamined Patent Application, First Publication No. 2005-317395 in the technical field of a wiring material for forming a circuit pattern and the like, a conductive coating film including a resin binder containing a metal nanowire having a long axis of 400 nm or more and a short axis of 50 nm or less is described, which film is used for wiring material in a circuit.

### SUMMARY

A conductive adhesion preventing film for medical use includes: a nonconductive base material; and a linear conductor (or, a conductor having a liner shape) having a length of 10 μm or more and a diameter (or, the longest length) of more than 50 nm and contained in the conductive adhesion preventing film by an amount of 5% by mass or more and 40% by mass or less. The conductive adhesion preventing film is formed on an electrode surface of a medical device performing at least one of incision, resection, coagulation, and ablation on living tissue by applying a high frequency voltage.

The nonconductive base material may include at least one material selected from a silica-based material, a silicone-based material, and a fluorine-based material.

The linear conductor may have a length of 10 μm or more and 200 μm or less and a diameter (or, the longest width) of more than 50 nm and 200 nm or less.

The conductive adhesion preventing film for medical use may further include conductive particles having a particle diameter of 15 μm or less. The linear conductor may be contained in the conductive adhesion preventing film by 5% by mass or more and 30% by mass or less, and the conductive particles may be contained in the conductive adhesion preventing film by 10% by mass or less.

A particle size of the conductive particles may be 0.5 μm or more and 15 μm or less, and the conductive particles may be contained in the conductive adhesion preventing film by 3% by mass or more and 10% by mass or less.

A surface of the conductive particles may be made of any one of silver, nickel, copper, and gold.

The conductive particles includes: a particle body made of a nonconductive substance; and a metal layer laminated on a surface of the particle body.

A medical device includes a conductive adhesion preventing film for medical use. The conductive adhesion preventing film includes: a nonconductive base material; and a linear conductor having a length of 10 μm or more and a diameter of more than 50 nm and contained in the conductive adhesion preventing film by an amount of 5% by mass or more and 40% by mass or less. The conductive adhesion preventing film is formed on an electrode surface of a medical device performing at least one of incision, resection, coagulation, and ablation on living tissue by applying a high frequency voltage.

### BRIEF DESCRIPTION OF THE DRAWINGS

FIG. 1 is a schematic configuration diagram showing an example of a medical device according to an embodiment of the present invention.

FIG. 2 is a sectional view taken along line AA in FIG. 1.

FIG. 3 is a schematic cross-sectional view of a conductive adhesion preventing film for medical use according to an embodiment of the present invention.

FIG. 4 is a schematic cross-sectional view showing a film structure of a first comparative example.

FIG. 5 is a schematic cross-sectional view showing a film structure of a second comparative example.

FIG. 6 is a schematic cross-sectional view showing a film structure of a third comparative example.

FIG. 7 is a schematic cross-sectional view of a conductive adhesion preventing film for medical use according to a first modification of the embodiment of the present invention.

FIG. 8 is a schematic cross-sectional view of a conductive adhesion preventing film for medical use according to a second modification of the embodiment of the present invention.

### DETAILED DESCRIPTION OF THE PREFERRED EMBODIMENTS

Hereinafter, a conductive adhesion preventing film for medical use and a medical device according to embodiments of the present invention will be described with reference to the drawings.

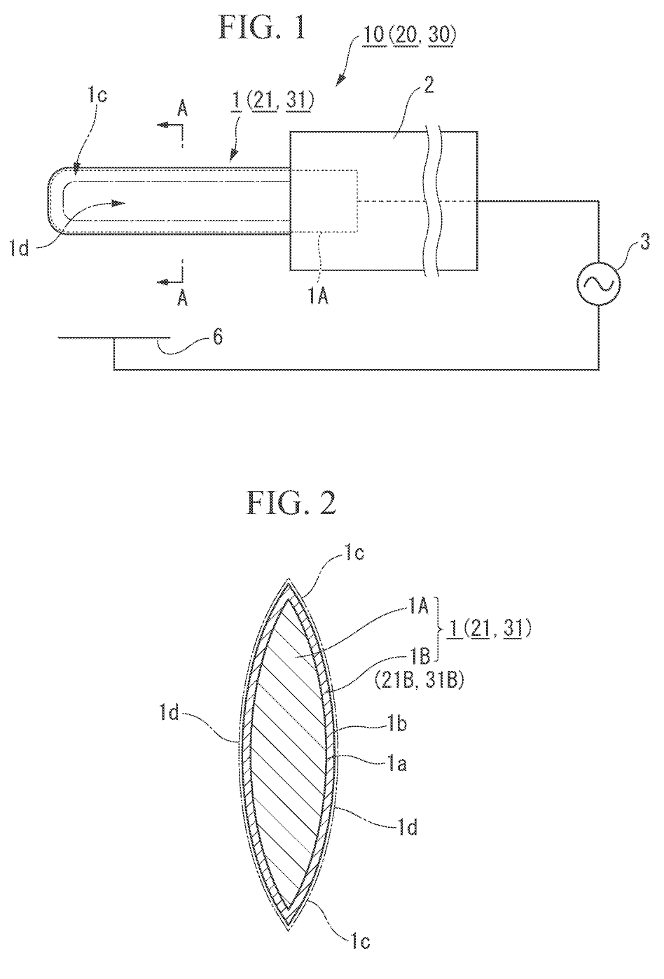

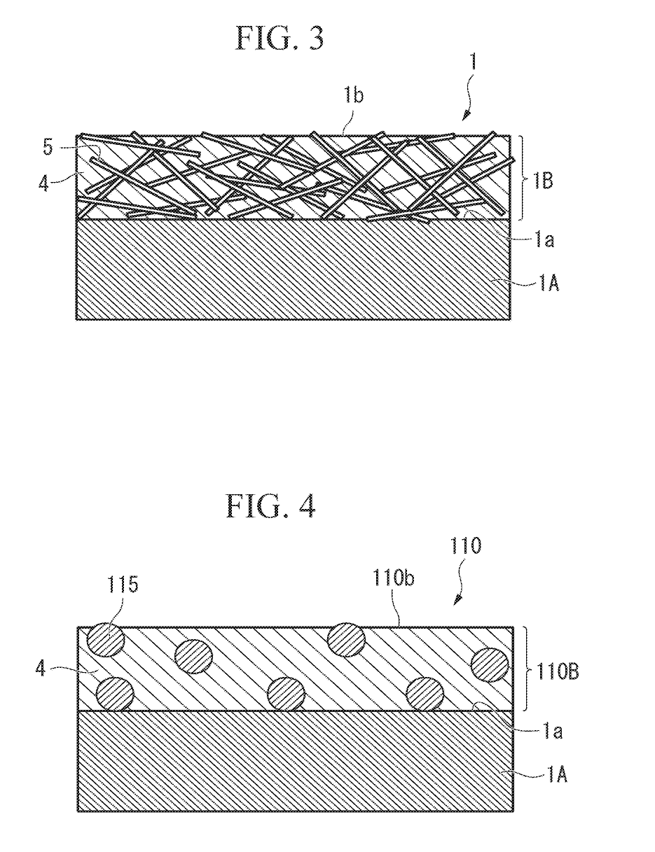

FIG. 1 is a schematic configuration diagram showing an example of a medical device according to an embodiment of the present invention. FIG. 2 is a sectional view taken along the line A-A in FIG. 1. FIG. 3 is a schematic cross-sectional view of a conductive adhesion preventing film for medical use according to an embodiment of the present invention.

The high frequency knife 10 of the present embodiment shown in FIG. 1 is an example of the medical device of the present embodiment. The high-frequency knife 10 is a medical treatment instrument that incises and cuts out a living tissue by applying a high-frequency voltage, or coagulates (stops blood vessel) or cauterizes a living tissue.

The high-frequency knife 10 includes a bar-shaped gripping portion 2 for the operator to hold with the hand and an electrode portion 1 protruding from the distal end of the grip portion 2.

The electrode unit 1 is brought into contact with the living tissue as the object to be treated and applies a high-frequency voltage. As shown in FIG. 2, the electrode portion 1 includes a metallic electrode main body 1A and a conductive adhesion preventing film 1B (conductive adhesion preventing film for medical use) according to this embodiment.

As shown in FIG. 1, the outer shape of the electrode main body 1A is a rectangular piece shape having a rounded corner portion at the tip in the projecting direction. As shown in FIG. 2, in the cross section orthogonal to the protruding direction, the electrode main body 1A has a flat shape in which the thickness decreases toward the outer edge. Although not specifically shown, the cross sectional shape of the electrode main body 1A at the tip portion in the protruding direction is also the same, and the thickness thereof becomes thin toward the outer edge.

As shown in FIG. 1, the electrode main body 1A has a base end portion held by the grip portion 2. The electrode main body 1A is electrically connected to the high-frequency power supply 3 by wiring connected to the base end portion of the electrode main body 1A. The high-frequency power source 3 is electrically connected to a counter electrode plate 6 to be attached to the object to be treated.

As shown in FIG. 2, the conductive adhesion preventing film 1B is a thin film provided so as to cover the electrode main body surface 1a. The outer surface of the conductive adhesion preventing film 1B constitutes the electrode surface 1b of the electrode portion 1.

An abdominal portion 1d formed in a gently curved shape or a planar shape as a whole is formed on the side portion of the electrode surface 1b excluding the blade portion 1c. The abdominal portion 1d is mainly used for performing treatment such as coagulation and cauterization by pressing the object to be treated.

As a material of the electrode main body 1A, an appropriate metallic material having conductivity such as a metal or an alloy is used. For example, an aluminum alloy, stainless steel, copper, or the like may be used as the material of the electrode main body 1A.

As schematically shown in FIG. 3, the conductive adhesion preventing film 1B includes a base material 4 (nonconductive base material) and a linear conductor 5 dispersed in the base material 4. A part of the linear conductor 5 is exposed to the outside from the base material 4. The surface of the base material 4 and the linear conductor 5 exposed from the surface of the base material 4 constitute the electrode surface 1b.

The film thickness of the conductive adhesion preventing film 1B can be set to an appropriate thickness to obtain strength necessary for the high frequency knife 10. For example, the thickness of the conductive adhesion preventing film 1B may be about 5 μm.

As the base material 4, it is possible to use a nonconductive material that is resistant to adhesion with living tissue and has heat resistance to withstand the heat generated during use of the high-frequency knife 10. The base material 4 may have a lower thermal conductivity than the linear conductor 5 described later. In this case, the base material 4 is also excellent in heat insulating performance.

For example, the base material 4 may be a material containing at least one of a silica-based material, a silicone-based material, and a fluorine-based material.

The linear conductor 5 has a linear shape with a length of 10 μm or more and a diameter exceeding 50 nm. The diameter of the linear conductor 5 is more preferably 70 nm or more.

The length and the diameter of the linear conductor 5 are measured by cutting the conductive adhesion preventing film 1B to make a cross section of the conductive adhesion preventing film 1B appear and observing the linear conductor 5 on the appeared surface with an electron microscope. Ion milling processing may be used as cross section processing.

As in the example shown in FIG. 3, one linear conductor 5 may have a constant diameter in the length direction or may have a diameter that varies in the length direction. When the diameter varies in the longitudinal direction, it suffices that the maximum diameter of one linear conductor 5 satisfies the above numerical range.

In one linear conductor 5, the diameter that varies in the length direction may be, for example, 10% or more and 100% or less of the maximum diameter. One linear conductor 5 may have a substantially constant diameter in the length direction.

The aspect ratio defined by the length/maximum diameter of the individual linear conductors 5 may be 50 or more and 4000 or less. The aspect ratio of each individual linear conductor 5 is more preferably 200 or more and 1000 or less.

Since FIG. 3 is a schematic view, the linear conductor 5 is drawn so as to extend straight. However, the linear conductor 5 is not limited to a straight shape as long as it can be satisfactorily dispersed in the conductive adhesion preventing film 1B. The linear conductor 5 may be curved or bent as long as it can be arranged in the range of the film thickness of the conductive adhesion preventing film 1B in the dispersed state in the conductive adhesion preventing film 1B.

The material of the linear conductor 5 may be metal. The metal used for the linear conductor 5 is preferably as low as the electrical resistivity. Examples of metals having low electrical resistivity include silver, nickel, copper, gold, and the like. Especially, nickel and copper are more preferable because they are inexpensive compared with silver, gold and the like.

However, the linear conductor 5 is not limited to metal as long as it has conductivity.

For example, as the linear conductor 5, a composite material of a linear nonconductive substance and a metal provided on the surface of the nonconductive substance may be used. In this case, it is more preferable that the metal covers the entire surface of the nonconductive substance.

Examples of the material of the nonconductive substance include inorganic materials such as glass, silica, alumina and zirconia. As a material of the nonconductive substance in the composite material, a resin material having heat resistance to withstand heat generated when using the high-frequency knife 10 may be used.

The nonconductive substance may have a hollow structure. In the case where the nonconductive substance has a hollow structure, the heat insulating property of the linear conductor 5 can be improved.

Examples of the metal in the composite material include silver, nickel, copper, gold, and the like. These metals may be coated on the surface of the non-conductive material. As a coating method, methods such as electroless plating, PVD (Physical Vapor Deposition), CVD (Chemical Vapor Deposition) and the like can be applied. Examples of PVD include sputtering, vapor deposition and the like.

In the case where the linear conductor 5 is formed of a composite material of a nonconductive substance and a metal, since the amount of expensive metal used is reduced as compared with the nonconductive substance, the cost of parts of the linear conductor 5 is reduced as compared with the case where the linear conductor 5 is made of metal alone.

For example, a non-metallic conductor may be used as the linear conductor 5. Carbon fiber, carbon nanotube, or the like may be used as the nonmetal conductor.

The linear conductor 5 is contained in the conductive adhesion preventing film 1B by 5% by mass or more and 40% by mass or less.

When the amount of the linear conductor 5 is less than 5% by mass, the probability that the linear conductors 5 come into contact with each other in the conductive adhesion preventing film 1B decreases, so that the number of the conductive paths due to the contact between the linear conductors 5 is reduced. In this case, good conductivity cannot be obtained in the conductive adhesion preventing film 1B.

When the amount of the linear conductor 5 exceeds 40% by mass, the area of the linear conductor 5 exposed on the electrode surface 1b becomes too wide and the interval between the exposed portions becomes too narrow. As a result, on the electrode surface 1b, the surface area of the base material 4 having a high adhesion preventing performance to biological tissues is narrowed, so that the adhesion preventing performance on the electrode surface 1b deteriorates.

The length of the linear conductor 5 is more preferably 10 μm or more and 200 μm or less.

If the length of the linear conductor 5 is less than 10 μm, the probability of contact with another linear conductor 5 decreases in the longitudinal direction of one linear conductor 5. In this case, good conductivity cannot be obtained in the conductive adhesion preventing film 1B.

In the case where the length of the linear conductor 5 exceeds 200 μm, when the conductive adhesion preventing film 1B is formed by coating, depending on the coating means, coating becomes difficult or uniform coating becomes difficult.

For example, in the case of forming the conductive adhesion preventing film 1B by spray coating, when the length of the linear conductor 5 exceeds 200 μm, clogging may occur in the spray part. For example, when the conductive adhesion preventing film 1B is formed by dip coating, the linear conductor 5 in the paint is apt to settle easily, making uniform coating difficult.

In order to further improve conductivity and paintability, it is more preferable that the length of the linear conductor 5 is 40 μm or more and 150 μm or less.

The diameter of the linear conductor 5 is more than 50 nm and more preferably 200 nm or less.

If the diameter of the linear conductor 5 is 50 nm or less, the cross-sectional area of the cross section orthogonal to the longitudinal direction becomes too small, so the strength decreases. In this case, when the high-frequency knife 10 is repeatedly used, the linear conductor 5 tends to be broken because it is repeatedly subjected to friction with living tissue or stress generated during use. When the linear conductor 5 is broken, the conductive path formed by the linear conductor 5 is cut, so that the conductivity of the conductive adhesion preventing film 1B is reduced. That is, the durability of the conductive adhesion preventing film 1B is reduced.

When the diameter of the linear conductor 5 exceeds 200 nm, uniform coating becomes difficult depending on the coating means when the conductive adhesion preventing film 1B is formed by coating.

For example, when the conductive adhesion preventing film 1B is formed by spray coating or dip coating, the linear conductor 5 in the coating easily becomes sedimented, and uniform coating becomes difficult.

In order to further improve the durability and coating property of the conductive adhesion preventing film 1B, the diameter of the linear conductor 5 is more preferably 70 nm or more and 150 nm or less.

The conductive adhesion preventing film 1B having the above-described structure may be formed by coating, for example. In this case, first, a coating material in which the base material 4 and the linear conductor 5 are dispersed is produced in an appropriate dispersion liquid such as water. Thereafter, this coating material is applied to the electrode main body surface 1a of the electrode main body 1A by appropriate coating means. The coating means is not particularly limited.

Examples of the coating means include spray coating, dip coating, spin coating, screen printing, inkjet printing, flexographic printing, gravure printing, pad printing, hot stamping and the like. Spray coating and dip coating are particularly suitable as coating means for forming the conductive adhesion preventing film 1B in a medical device because they can be applied easily even if the shape of the object to be coated is complicated.

When the paint is applied to the electrode main body surface 1a of the electrode main body 1A, the linear electric conductor 5 moves within the paint until the paint is dried. At this time, the linear electric conductor 5 in the paint is oriented along the electrode main body surface 1a which is a coated surface by an external force or gravity acting from a coating means at the time of coating. That is, the linear conductor 5 in the paint is mixed with the base material 4 and is also entangled with other linear conductors 5, and it tends to take a posture in which the linear conductors 5 intersect with the electrode main body surface 1a in parallel or shallow angle.

After the coating film is formed on the electrode main body surface 1a, drying is performed, whereby the dispersion liquid is evaporated. As a result, the conductive adhesion preventing film 1B in which the linear conductor 5 is dispersed in the base material 4 is formed.

As schematically shown in FIG. 3, inside the conductive adhesion preventing film 1B, the linear conductor 5 is dispersed so as to intersect with the electrode main body surface 1a parallel or shallowly. The linear conductor 5 is also dispersed in an attitude that intersects with the electrode surface 1b formed at substantially the same distance from the electrode main body surface 1a at the same angle.

In the conductive adhesion preventing film 1B, most of the linear conductors 5 are in contact with other linear conductors 5. An end portion or a side portion of the linear conductor 5 in the vicinity of the electrode main body surface 1a is in contact with the electrode main body surface 1a.

The end portion or the side portion of the linear conductor 5 near the surface of the base material 4 is exposed from the surface of the base material 4 constituting a part of the electrode surface 1b. Here, since the probability that the linear conductor 5 becomes parallel to the surface of the base material 4 is much smaller than in the case where it is not parallel, in most cases, what is exposed to the outside of the base material 4 is an end portion of the linear conductor 5.

The protrusion amount of the linear conductor 5 exposed from the surface of the base material 4 may be 0.1 nm or more and 500 nm or less.

The exposed area of the exposed portion of the linear conductor 5 in plan view varies depending on the amount of protrusion of the linear conductor 5 and the tilted state of the linear conductor 5, but generally it is less than the cross-sectional area of the linear conductor 5 cut by a plane parallel to the electrode main body surface 1a.

Next, the operation of the high-frequency knife 10 having the above-described configuration will be described.

The treatment using the high-frequency knife 10 is performed, for example, in a state in which the counter electrode plate 6 is attached to the patient and a high frequency voltage is applied to the electrode unit 1 by the high frequency power supply 3. The operator brings the blade portion 1c or the abdominal portion 1d of the electrode portion 1 into contact with the object to be treated such as the treated portion of the patient while applying the high-frequency voltage to the electrode portion 1.

The electrode part 1 is covered with a conductive adhesion preventing film 1B. In the inside of the conductive adhesion preventing film 1B, the linear conductors 5 are dispersed. A large number of linear conductors 5 are dispersed in contact with each other inside the conductive adhesion preventing film 1B. For this reason, most of the linear conductors 5 are directly or indirectly connected to the electrode main body surface 1a. That is, inside the conductive adhesion preventing film 1B, a plurality of conductive members 5 which are in contact with each other and which conduct the end portion of the linear conductor 5 constituting a part of the electrode surface 1b and the electrode main body surface 1a, thereby the conductive paths are formed.

The electrode surface 1b of the conductive adhesion preventing film 1B is formed of a smooth surface of the base material 4 except for the linear conductor 5 exposed from the base material 4. The area in plan view of the exposed portion of the linear conductor 5 is much smaller than the surface area of the base material 4. The amount of protrusion of the exposed portion of the linear conductor 5 from the surface of the base material 4 is also small.

When a high frequency voltage is applied between the electrode part 1 and the counter electrode plate 6, a high frequency current is generated via the conductive adhesion preventing film 1B. Since the conductive portion at the contact portion between the electrode surface 1b of the electrode portion 1 and the living tissue is the exposed portion of the linear conductor 5, it is much smaller than the electrode area of the counter electrode plate 6. Therefore, at the contact portion between the electrode portion 1 and the living tissue, a current having a large current density flows from the linear conductor 5 exposed at the electrode surface 1b to the living tissue, and Joule heat is generated. As a result, the moisture of the living tissue of the object to be treated rapidly evaporates, and the living tissue is ruptured by the blade portion 1c. Therefore, by moving the electrode unit 1 with respect to the living tissue, it is possible to incise and cut the living tissue.

When a high-frequency current is applied in a state where the abdomen 1d is pressed against the object to be treated, moisture in the living tissue of the object rapidly evaporates, and the living tissue is coagulated in the vicinity of the abdominal portion 1d. Therefore, when the abdomen 1d is pressed against the object to be treated, hemostasis and cauterization of living tissue can be performed.

When the necessary treatment is completed, the operator separates the electrode unit 1 from the object to be treated. At this time, the majority of the electrode surface 1b in contact with the living tissue is not the linear electric conductor 5 to which the living tissue is likely to adhere, but the base material 4 to which the living tissue hardly adheres. Therefore, at the time of separating the electrode portion 1, the living tissue easily peels off from the electrode surface 1b.

Further, the electrode surface 1b is a roughened surface in which minute protrusions are formed by the exposed portion of the linear conductor 5. Therefore, as compared with the case where the electrode surface 1b is composed only of a smooth surface like the surface of the base material 4, the adhesion of the living tissue is weakened. Also in this respect, when separating the electrode portion 1, the living tissue easily peels off from the electrode surface 1b.

As described above, in the high-frequency knife 10, the living tissue hardly adheres to the electrode surface 1b.

If the biological tissue that cannot be peeled off adheres to the electrode surface 1b, the conductivity at the adhered portion is lowered, so that the electric energy is not sufficiently released from the adhered portion. Therefore, the treatment performance is deteriorated in the attachment portion of the living tissue.

However, as described above, since the living tissue hardly adheres to the electrode surface 1b of the electrode unit 1, the high-frequency knife 10 can prevent deterioration in the treatment performance during treatment. Furthermore, even when the electrode portion 1 is repeatedly used, the durability of the electrode portion 1 is secured.

Since the linear conductor 5 in the conductive adhesion preventing film 1B has a length of 10 μm or more and a diameter exceeding 50 nm, it is thicker than a metal nanowire that is proposed to add for the purpose of improving the dispersibility in the conductive coating film. Therefore, the conductive path formed by the linear conductor 5 is hardly broken by external force and stress acting on the conductive adhesion preventing film 1B at the time of treatment. Therefore, the durability of the conductive adhesion preventing film 1B is improved as compared with the case where conductivity is imparted by the metal nanowires.

Here, the conductive path formed in the conductive adhesion preventing film 1B will be described in detail in comparison with the first to third comparative examples.

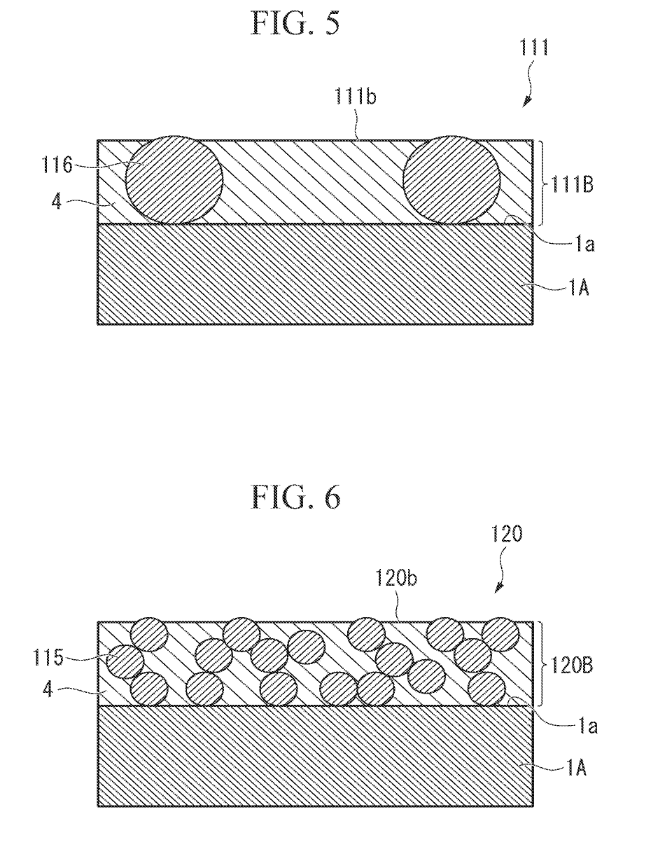

FIG. 4 is a schematic cross-sectional view showing the film structure of the first comparative example. FIG. 5 is a schematic cross-sectional view showing the film structure of the second comparative example. FIG. 6 is a schematic cross-sectional view showing the film structure of the third comparative example.

### First Comparative Example

The electrode unit 110 in the first comparative example schematically shown in FIG. 4 includes a film 110B in place of the conductive adhesion preventing film 1B of the electrode unit 1 of the present embodiment.

The film 110B includes metal particles 115 instead of the linear conductor 5 of the present embodiment. The particle diameter of the metal particles 115 in this comparative example is less than the thickness of the base material 4 in the film 110B.

Regarding the amount of the metal particles 115 in the film 110B, the amount is adjusted so that the exposed area and the exposed density in plan view of the metal particles 115 in the electrode surface 110b are substantially the same as the exposed area and exposure density of the linear conductor 5 of the present embodiment.

The metal particles 115 disperse substantially uniformly inside the coating material forming the film 110B. If the distribution of the metal particles 115 on the surface of the coating film is rough, the distribution inside the coating film is likewise rough. Therefore, as schematically shown in FIG. 4, the metal particles 115 inside the film 110B are in a state where they are hardly in contact with each other. Therefore, in the metal particle 115, almost no conductive path is formed.

As described above, the film 110B in this comparative example has adhesion prevention performance similar to that in the present embodiment. However, in this comparative example, no conductive path is formed in which the metal particles 115 are in contact with each other inside the film 110B, so that the conductivity of the film 110B cannot be obtained.

### Second Comparative Example

The electrode unit 111 in the second comparative example schematically shown in FIG. 5 includes a film 111B instead of the film 110B in the first comparative example.

The film 111B includes metal particles 116 in place of the metal particles 115 of the film 110B in the first comparative example. The particle diameter of the metal particles 116 in this comparative example is slightly larger than the thickness of the base material 4 in the film 111B.

In this comparative example, each metal particle 116 abuts on the electrode main body surface 1a and projects beyond the surface of the base material 4 to form a part of the electrode surface 111b. Each metal particle 116 constitutes a conductive path. As a result, the film 111B has conductivity. The degree of conductivity depends on the number of metal particles 116.

The exposed area of the metal particle 116 in plan view depends on the thickness of the base material 4 in the film 111B. In order to reduce the exposed area of the metal particle 116 in plan view, the difference between the thickness of the base material 4 and the diameter of the metal particle 116 may be reduced. However, depending on the film thickness variation of the coating film, the metal particles 116 may be fully buried. If the difference between the thickness of the base material 4 and the diameter of the metal particle 116 is made too small, the conductivity is lowered.

Therefore, for example, when the target thickness of the base material 4 in the film 111B is 5 μm, the diameter of the metal particle 116 needs to be about 5.5 μm.

As described above, in this comparative example, the area of the exposed portion of the metal particle 116 per unit is significantly larger than in the first comparative example and the present embodiment described later. Therefore, in order to satisfy the adhesion prevention performance, it is necessary to sufficiently separate the spaces of the metal particles 116 in a plan view by reducing the amount of the metal particles 116 in the film 111B. However, when the amount of the metal particles 116 decreases, the number of the conductive paths also decreases, so that the conductivity decreases.

As described above, in this comparative example, there is a trade-off relationship between adhesion preventing performance and conductivity in the film 111B.

Further, in this comparative example, the metal particles 116 have a large mass and are coated so as to be in contact with the electrode surface 111b. Therefore, in the coating process, the metal particles 116 tend to roll on the electrode surface Illb by the action of external force. As a result, there is also a problem that distribution of the metal particles 116 in the coating film in a plan view is not uniform.

For example, when the metal particles 116 move during painting and a portion where the metal particles 116 are crowded together occurs, the surface area of the base material 4 relatively decreases at this portion, so that the biological tissue tends to adhere more easily.

### Third Comparative Example

The electrode unit 120 in the third comparative example schematically shown in FIG. 6 includes a film 120B instead of the film 110B in the first comparative example.

The film 120B is formed by increasing the amount of the metal particles 115 in the film 110B in the first comparative example. The amount of the metal particles 115 in the film 120B is adjusted to such an extent that a chain state of the metal particles 115 traversing in the thickness direction of the base material 4 is reliably formed.

The metal particles 115 disperse substantially evenly inside the coating material forming the film 120B. When a chain state of the metal particles 115 traversing in the thickness direction of the base material 4 is formed in the coating film, a similar chain state occurs between the metal particles 115 also in the direction along the electrode main body surface 1a. Therefore, as schematically shown in FIG. 6, in the electrode surface 120b of the film 120B, the exposed portions of the metal particles 115 are more densely distributed in plan view than in the first comparative example. Therefore, in the electrode surface 120b, the interval between the exposed portions of the metal particles 115 in the plan view is shortened, and the adhesion preventing performance is lowered as compared with the first comparative example.

As described above, although the film 120B in this comparative example can obtain good conductivity, adhesion prevention performance deteriorates.

In the first to third comparative examples described above, metal particles having an aspect ratio close to 1 are added to the base material 4, whereas in the conductive adhesion preventing film 1B of the present embodiment, the linear conductor 5 is added.

Since the linear conductor 5 has an aspect ratio greater than that of metal particles, the linear conductor 5 is oriented in a direction along the electrode main body surface 1a which is the coated surface of the coating film after coating. Specifically, the linear conductors 5 are oriented such that the longitudinal direction intersects the normal of the coated surface at an angle equal to or close to 90°.

For example, when the film thickness of the base material 4 in the conductive adhesion preventing film 1B is 5 μm, the minimum length of the linear conductor 5 is 10 μm. Therefore, when the linear conductor 5 is tilted by 30° relative to the electrode main body surface 1a (60° with respect to the normal to the electrode surface 1b), a single conductive path is formed which conducts from the electrode main body surface 1a to the electrode surface 1b by one linear conductor 5. If the length of the linear conductor 5 is doubled, a conductive path is formed by one linear conductor 5 tilted by 14.5° relative to the electrode main body surface 1a (about 75.5° with respect to the normal to the electrode surface 1b).

If the tilt of the linear conductor 5 with respect to the electrode main body surface 1a is half of that described above, two or more linear conductors 5 abut each other, so that one conductive path is formed. Since the contact portions of the respective linear conductors 5 may be any positions in the longitudinal direction of the respective linear conductors 5, in the above-described example, there is a possibility of contact in the range of 10 μm and 20 μm, respectively.

The diameter of the linear conductor 5 may be appropriately larger than 50 nm, and may be more than 50 nm and 200 nm or less. Therefore, in the longitudinal direction of 10 μm or more, it is possible to make contact with many other linear conductors 5 in an intersecting manner.

For this reason, as schematically shown in FIG. 3, the linear conductors 5 in the conductive adhesion preventing film 1B are arranged in a net shape by abutting each other. In this way, by using the linear conductor 5, even if the amount of the linear conductors 5 in the conductive adhesion preventing film 1B is low, good conductivity can be obtained.

The exposed area of each of the linear conductors 5 on the electrode surface 1b is less than the cross sectional area of the linear conductor 5 cut by the plane parallel to the electrode main body surface 1a. Therefore, it is easy to make the exposed area per single linear conductor 5 smaller than the exposed area of the metal particles in each of the above-described comparative examples. For this reason, living tissue hardly adheres to the exposed portion of the linear conductor 5.

In the case of metal particles, it is easy to densely collect the metal particles, so that the interval in the plan view of the exposed portions of the metal particles can easily approach the particle size. On the other hand, since the linear conductor 5 has a reticulate shape sterically intertwined within the conductive adhesion preventing film 1B, the possibility that the exposed portions of the linear conductor 5 are densely collected is much less than the possibility of the exposed portion of the metal particle densely gathering together

Therefore, the adhesion preventing performance of the living body tissue on the conductive adhesion preventing film 1B is improved, even at the point where the spaces between the exposed portions of the linear electric conductor 5 are likely to be separated.

As described above, in the conductive adhesion preventive film 1B of the present embodiment, by including the linear conductor 5 in an appropriate amount in the base material 4, it is possible to achieve both the conductivity and the adhesion preventing performance to the living body tissue in the conductive adhesion preventing film 1B.

That is, by adjusting the length of the linear conductor 5, the probability that the linear conductors 5 contact each other within the coating film and the probability that the end of the linear conductor 5 contacts the electrode main body surface 1a can be increased, thereby it is possible to increase the conductivity.

Since the conductivity can be secured by adjusting the length of the linear conductor 5, the diameter of the linear conductor 5 can be reduced within a range where the required strength can be obtained. For example, even if the diameter of the linear conductor 5 is about ½ to 1/2000 of the thickness of the base material 4, good conductivity can be obtained.

As a result, the amount of the linear conductor 5 can be reduced.

In this way, since the diameter of the linear conductor 5 can be reduced, the size of the exposed portion of the linear conductor 5 on the electrode surface 1b in the plan view can be reduced. Furthermore, since the linear conductors 5 are entangled with each other and dispersed in the base material 4, it becomes easy to separate the exposed portions of the linear electric conductor 5 in plan view. As a result, the conductive adhesion preventive film 1B has good adhesion preventing performance of living tissue.

On the other hand, in the first to third comparative examples, only metal particles are used to obtain conductivity. When the diameter of the metal particles is determined, the volume and mass are determined, and the distribution in the coating film and the size of the exposed portion are limited depending on the content. Therefore, it is difficult to satisfy both conductivity and adhesion preventing performance of living tissue.

In the present embodiment, since the diameter and the length of the linear conductor 5 can be respectively changed, by combining these shape conditions and the content, the distribution in the coating film and the size of the exposed portion can be further finely adjusted.

As described above, according to the high-frequency knife 10 of the present embodiment, since the conductive adhesion preventing film 1B is provided on the surface of the electrode portion 1, it is difficult for the living tissue to adhere even if it is repeatedly used for the treatment of living tissue, and the conductivity can be kept satisfactory. Therefore, the high-frequency knife 10 is excellent in durability.

### First Modification Example

A conductive adhesion preventing film for medical use and a medical device according to a first modification of the present embodiment will be described.

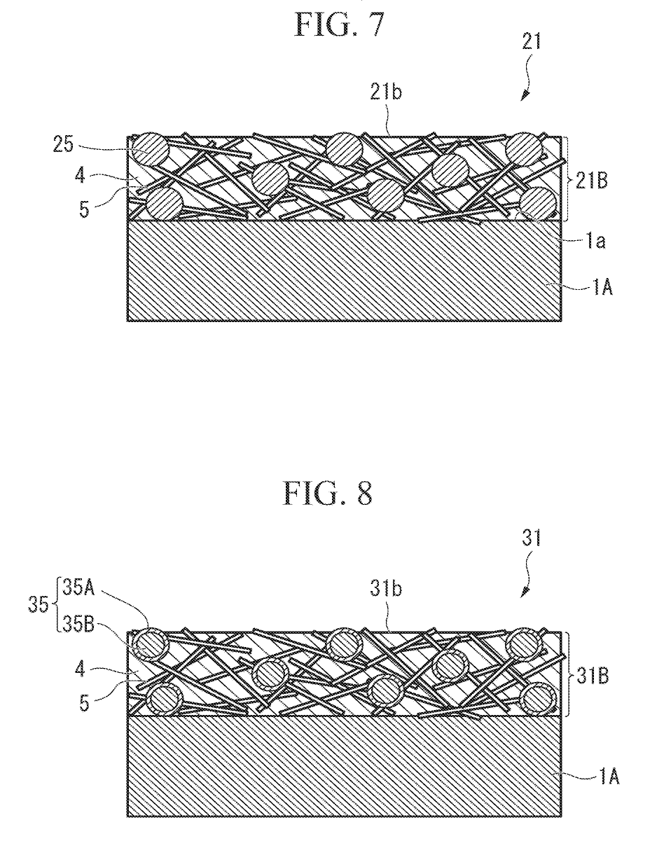

FIG. 7 is a schematic cross-sectional view of a conductive adhesion preventing film for medical use of a first modification of the embodiment of the present invention.

As shown in FIG. 1, the high frequency knife 20 (medical device) of this modification includes an electrode portion 21 instead of the electrode portion 1 in the above embodiment. As shown in FIG. 2, in place of the conductive adhesion preventing film 1B of the electrode portion 1 in the above-described embodiment, the electrode portion 21 in the present modification includes the conductive adhesion preventing film 21B (conductive adhesion preventing film for medical use).

Hereinafter, differences from the above embodiment will be mainly described.

As schematically shown in FIG. 7, the conductive adhesion preventing film 21B has the same base material 4 and linear conductor 5 as the conductive adhesion preventing film 1B in the above embodiment, and further has a conductive particles 25 of a particle diameter of 15 μm or less. As a method for measuring the particle diameter of the conductive particles 25, a light scattering type particle size analyzer is used. Specifically, depending on the distribution range of the particle size of the measurement target, for example, a Microtrack particle size analyzer based on a laser diffraction/scattering method, a Nanotrac particle size analyzer based on a dynamic light scattering method, or the like can be appropriately used. The maximum diameter/minimum diameter value of each individual conductive particle 25 may be 1 or more and 10 or less.

However, the linear conductor 5 is contained in the conductive adhesion preventing film 21B by 5% by mass or more and 30% by mass or less, and the conductive particles 25 is contained in the conductive adhesion preventing film 21B by more than 0% by mass and equal to or less than 10% by mass.

The conductive particles 25 are made of metal particles. The metal used for the conductive particles 25 is preferably as low as the electrical resistivity. Examples of metals having low electrical resistivity include silver, nickel, copper, gold, and the like. Especially, nickel and copper are more preferable because they are inexpensive compared with silver, gold and the like.

Like the conductive adhesion preventing film 1B of the above embodiment, the conductive adhesion preventing film 21B may be formed by coating. For example, a coating material in which the base material 4, the linear conductor 5, and the conductive particles 25 are dispersed is produced in a dispersion liquid such as water. Thereafter, this coating material is applied to the electrode main body surface 1a of the electrode main body 1A by the same coating means as in the above embodiment.

After the coating film is formed on the electrode main body surface 1a, drying is performed, whereby the dispersion liquid is evaporated. As a result, the conductive adhesion preventing film 21B in which the linear conductor 5 and the conductive particles 25 are dispersed is formed on the base material 4.

The dispersion state of the linear conductor 5 in the conductive adhesion preventing film 21B is the same as in the above embodiment. However, the conductive particles 25 whose content is 10% by mass or less are also dispersed substantially evenly in the conductive adhesion preventing film 21B. Therefore, most of the conductive particles 25 are dispersed in the base material 4 in a state of being separated from each other, and a part thereof is in contact with the linear conductor 5 and the electrode main body surface 1a. A part of the conductive particles 25 located in the vicinity of the surface of the base material 4 is exposed to the outside from the surface of the base material 4.

The linear conductor 5 and the conductive particles 25 exposed from the surface of the base material 4 and the surface of the base material 4 constitute the electrode surface 21b.

The conductive particles 25 inside the conductive adhesion preventing film 21B and in contact with the linear conductor 5 constitute a part of the conductive path. Therefore, as the amount of the conductive particles 25 increases, the electrical resistivity decreases, so that the conductivity of the conductive adhesion preventing film 21B is improved.

The conductive particles 25 exposed to the outside from the base material 4 form a convex shape on the electrode surface 21b together with the exposed linear conductor 5. That is, the conductive particles 25 exposed to the outside from the base material 4 have the function of roughening the electrode surface 21b like the linear conductor 5.

In the case where the conductive particles 25 exposed from the base material 4 are in contact with the linear conductor 5 which is in electrical conduction with the electrode main body surface 1a inside the base material 4, the exposed portion of the conductive particle 25 conducts to the electrode main body surface 1a via the linear conductor 5. In this case, the exposed portion of the conductive particles 25 constitutes a conductive portion on the electrode surface 21b.

When the particle size of the conductive particles 25 exceeds 15 μm, the height of the portion exposed from the surface of the base material 4 becomes too large. Further, the exposed area of the exposed portion of the conductive particle 25 in plan view becomes too large.

When the convex shape on the electrode surface 21b becomes too high, the biological tissue is strongly pressed against the convex conductive particles 25 more than the concave base material 4, so that the living tissue easily adheres to the conductive particles 25. Therefore, the adhesion preventing performance of the conductive adhesion preventing film 21B is deteriorated. Furthermore, even at the point that the exposed area of the exposed portion of the conductive particle 25 in the plan view becomes too large, the living tissue tends to adhere to the exposed portion of the conductive particle 25.

Furthermore, if the convex shape on the electrode surface 21b becomes too high, stress tends to concentrate on the convex portion when the living tissue comes into contact with the electrode surface 21b, so that the durability of the conductive adhesion preventing film 21B also decreases.

When the particle diameter of the conductive particles 25 is reduced, the volume of the conductive particles 25, the height of the convex shape, and the exposed area are also decreased, so that the function of improving the conductivity and adhesion preventing performance also decreases. In order to further improve the conductivity and adhesion preventing performance of the conductive adhesion preventing film 21B, it is more preferable that the particle size of the conductive particles 25 is 0.5 μm or more.

When the amount of the conductive particles 25 exceeds 10% by mass, the exposure amount of the conductive particles 25 becomes too large, so that the adhesion preventing performance is lowered.

On the other hand, when the amount of the conductive particles 25 decreases, the exposed amount of the conductive particles 25 becomes too small, and the protrusion height and the exposed area of the conductive particles 25 decrease. For this reason, the conductivity and adhesion preventing property are deteriorated. In order to further improve the conductivity and adhesion preventing performance of the conductive adhesion preventing film 21B, the amount of the conductive particles 25 is more preferably 3% by mass or more.

According to the high-frequency knife 20 of this modification, since the conductive adhesion preventing film 21B is provided on the surface of the electrode portion 21, it is difficult for living tissue to adhere to it even when it is repeatedly used for the treatment of living tissue, and the conductivity can be keep favorably. Therefore, the high-frequency knife 20 is excellent in durability.

Particularly, in this modified example, since the conductive particles 25 are included in addition to the linear conductor 5, the convex shape on the electrode surface 21b is changed to a more varied shape, so that the adhesion preventing property is further improved.

Since the dispersibility of the conductive particles 25 in the coating material is better than that of the linear conductor 5, even if the coating method is difficult to coat when the amount of the linear conductor 5 is increased, the coating is easy.

### Second Modification

A conductive adhesion preventing film for medical use and a medical device according to a second modification of the present embodiment will be described.

FIG. 8 is a schematic cross-sectional view of a conductive adhesion preventing film for medical use according to a second modification of the embodiment of the present invention.

As shown in FIG. 1, the high-frequency knife 30 (medical device) of this modification includes an electrode portion 31 instead of the electrode portion 21 in the first modification. As shown in FIG. 2, in place of the conductive adhesion preventing film 21B of the electrode portion 21 in the first modification, the electrode portion 31 in this modification is formed by laminating the conductive adhesion preventing film 31B (conductive adhesion preventing film for medical use).

Hereinafter, differences from the first modified example will be mainly described.

As schematically shown in FIG. 8, the conductive adhesion preventing film 31B includes conductive particles 35 instead of the conductive particles 25 in the first modification. The conductive particles 35 exposed to the outside from the base material 4 form a convex shape on the electrode surface 31b together with the exposed linear conductor 5.

The conductive particle 35 includes a particle main body 35B made of a nonconductive substance and a metal layer 35A laminated on the surface of the particle main body 35B. The particle size of the conductive particles 35 is the same as that of the conductive particles 25 in the first modification. The amount of the conductive particles 35 may be the same as that of the conductive particles 25 in the first modification. However, depending on the mass of the particle main body 35B, the same conductivity can be obtained with a smaller amount of the conductive particles 35 than of the conductive particles 25. Therefore, within a range where necessary adhesion prevention performance can be obtained, the amount of the conductive particles 35 may be different from the amount of the conductive particles 25.

Examples of the material of the nonconductive substance constituting the particle main body 35B include inorganic materials such as glass, silica, alumina, and zirconia. As the material of the particle main body 35B, a resin material having heat resistance to withstand heat generated when the high-frequency knife 30 is used may be used.

Although FIG. 8 shows an example in which the particle body 35B is a solid body, the particle body 35B may have a hollow structure. The hollow structure may be a spherical shell structure or a porous structure. When the particle main body 35B has a hollow structure, the heat insulating property of the conductive particles 35 can be improved.

Examples of the material of the metal layer 35A include silver, nickel, copper, gold, and the like. These metals may be coated on the surface of the non-conductive material. As a coating method, methods such as electroless plating, PVD (Physical Vapor Deposition), CVD (Chemical Vapor Deposition) and the like can be applied. Examples of PVD include sputtering, vapor deposition and the like.

In the conductive particle 35, since the amount of metal used is reduced as compared with the conductive particle 25 having the same diameter in the first modification, the cost of parts can be reduced.

Since the conductive adhesion preventing film 31B uses conductive particles 35 having a particle diameter in the same range as that of the conductive particles 25 of the first modification and whose surface is covered with the metal layer 35A, as in the case of the first modified example, it is difficult for living tissue to adhere even if it is repeatedly used for treating living tissue, and the conductivity can be kept satisfactory. Therefore, the high-frequency knife 30 is excellent in durability.

In the above description of the embodiments and modifications, the medical device including the conductive adhesion preventing film for medical use is described as an example of a high frequency knife, but the medical device is not limited to a high frequency knife. Examples of other medical devices that can suitably use the conductive adhesion preventing film for medical use of the present invention include treatment devices such as an electric scalpel, a high frequency knife, a bipolar forceps, a probe, a snare, and the like.

In the above description of the embodiment and modifications, the conductive adhesion preventing film for medical use is directly laminated on the electrode main body 1A, but a single layer or multilayered intermediate layer having conductivity may be interposed between the electrode main body 1A and the conductive adhesion preventing film for medical use. As the intermediate layer, an appropriate conductive layer for improving the bonding strength between the electrode main body 1A and the conductive adhesion preventing film for medical use may be used.

### EXAMPLE

Next, Examples 1 to 16 of the conductive adhesion preventing film for medical use corresponding to the above-described embodiment and the first modified example will be described together with Comparative Examples 1 to 6. The schematic constitution and evaluation results of each example and comparative example are shown in the following Table 1.

TABLE 1

| | | | | | | CONDUCTIVE PARTICLES | |
|---|---|---|---|---|---|---|---|
| | | LINEAR CONDUCTOR | | | | | PARTICLE |
| | BASE | | LENGTH | DIAMETER | CONTENT | | DIAMETER |
| | MATERIAL | MATERIAL | (μm) | (nm) | (% BY MASS) | MATERIAL | (μm) |
| EXAMPLE 1 | SILICONE | Cu | 10 | 100 | 10 | — | — |
| EXAMPLE 2 | SILICONE | Ni | 200 | 200 | 10 | — | — |
| EXAMPLE 3 | SILICONE | Ag | 40 | 50 | 10 | — | — |
| EXAMPLE 4 | SILICONE | Ag | 40 | 70 | 10 | — | — |
| EXAMPLE 5 | SILICONE | Ag | 40 | 70 | 5 | — | — |
| EXAMPLE 6 | SILICONE | Ag | 40 | 70 | 40 | — | — |
| EXAMPLE 7 | FLUORINE | Ag | 40 | 70 | 10 | — | — |
| EXAMPLE 8 | SILICA | Ag | 40 | 70 | 10 | — | — |
| EXAMPLE 9 | SILICONE | Ag | 40 | 70 | 10 | Cu | 0.1 |
| EXAMPLE 10 | SILICONE | Ag | 40 | 70 | 10 | Cu | 0.5 |
| EXAMPLE 11 | SILICONE | Ag | 40 | 70 | 10 | Cu | 5.5 |
| EXAMPLE 12 | SILICONE | Ag | 40 | 70 | 10 | Cu | 8.2 |
| EXAMPLE 13 | SILICONE | Ag | 40 | 70 | 10 | Cu | 10.7 |
| EXAMPLE 14 | SILICONE | Ag | 40 | 70 | 10 | Cu | 5.5 |
| EXAMPLE 15 | SILICONE | Ag | 40 | 70 | 10 | Cu | 5.5 |
| EXAMPLE 16 | SILICONE | Ag | 40 | 70 | 10 | Cu | 5.5 |
| COMPARATIVE | SILICONE | Cu | 5 | 150 | 10 | — | — |
| EXAMPLE 1 | | | | | | | |
| COMPARATIVE | SILICONE | Ag | 35 | 40 | 10 | — | — |
| EXAMPLE 2 | | | | | | | |
| COMPARATIVE | SILICONE | Ag | 40 | 70 | 3 | — | — |
| EXAMPLE 3 | | | | | | | |
| COMPARATIVE | SILICONE | Ag | 40 | 70 | 50 | — | — |
| EXAMPLE 4 | | | | | | | |
| COMPARATIVE | SILICONE | — | — | — | 0 | Cu | 5.5 |
| EXAMPLE 5 | | | | | | | |
| COMPARATIVE | SILICONE | — | — | — | 0 | Cu | 5.5 |
| EXAMPLE 6 | | | | | | | |

| | CONDUCTIVE | EVALUATION | | | |
|---|---|---|---|---|---|
| | PARTICLES | ADHESION | | | |
| | CONTENT | PREVENTION | | | OVER-ALL |
| | (% BY MASS) | PROPERTY | CONDUCTIVITY | DURABILITY | EVALUATION |
| EXAMPLE 1 | 0 | ◯ | ◯ | ◯ | ◯ |
| EXAMPLE 2 | 0 | ◯ | ◯ | ◯ | ◯ |
| EXAMPLE 3 | 0 | ◯ | ◯ | X | X |
| EXAMPLE 4 | 0 | ◯ | ◯ | ◯ | ◯ |
| EXAMPLE 5 | 0 | ◯ | ◯ | ◯ | ◯ |
| EXAMPLE 6 | 0 | ◯ | ◯ | ◯ | ◯ |
| EXAMPLE 7 | 0 | ◯ | ◯ | ◯ | ◯ |
| EXAMPLE 8 | 0 | ◯ | ◯ | ◯ | ◯ |
| EXAMPLE 9 | 5 | ◯ | ◯ | ◯ | ◯ |
| EXAMPLE 10 | 5 | ⊚ | ◯ | ◯ | ⊚ |
| EXAMPLE 11 | 5 | ⊚ | ◯ | ◯ | ⊚ |
| EXAMPLE 12 | 5 | ⊚ | ◯ | ◯ | ⊚ |
| EXAMPLE 13 | 5 | ⊚ | ◯ | ◯ | ⊚ |
| EXAMPLE 14 | 1 | ◯ | ◯ | ◯ | ◯ |
| EXAMPLE 15 | 3 | ⊚ | ◯ | ◯ | ⊚ |
| EXAMPLE 16 | 10 | ⊚ | ◯ | ◯ | ⊚ |
| COMPARATIVE | 0 | ◯ | X | ◯ | X |
| EXAMPLE 1 | | | | | |
| COMPARATIVE | 0 | ◯ | ◯ | X | X |
| EXAMPLE 2 | | | | | |
| COMPARATIVE | 0 | ◯ | X | ◯ | X |
| EXAMPLE 3 | | | | | |
| COMPARATIVE | 0 | X | ◯ | ◯ | X |
| EXAMPLE 4 | | | | | |
| COMPARATIVE | 10 | ◯ | X | ◯ | X |
| EXAMPLE 5 | | | | | |
| COMPARATIVE | 50 | X | ◯ | ◯ | X |
| EXAMPLE 6 | | | | | |

### Example 1

Example 1 is an example of the conductive adhesion preventing film 1B of the above embodiment.

As shown in Table 1, a silicone resin (abbreviated as “silicone” in Table 1) was used as a material of the base material 4 (reference numerals are omitted in Table 1, the same applies below). As the silicone resin, SILRES (registered trademark) MPF 52 E (trade name; manufactured by Wacker Asahi Kasei Silicone Co., Ltd.) was used.

The linear conductor 5 contained 10% by mass of a copper (Cu) wire (manufactured by EM Japan Co., Ltd., the same applies to the following linear conductor) having a length of 10 μm and a diameter of 100 nm.

The conductive adhesion preventing film 1B was formed on the surface of an aluminum substrate (material: A 5052 P) made of a square plate of 50 mm×50 mm×3 mm. The film thickness of the conductive adhesion preventing film 1B was set to 5 μm.

In order to form the above-described conductive adhesion preventing film 1B, a coating material was prepared in which the copper wire and the silicone resin dispersed in water were mixed so that the amount of the copper wire having a length of 10 μm and a diameter of 100 nm became 10% by mass after the film formation. This coating material was applied onto the aluminum substrate by dip coating. At this time, part of the film was masked for film thickness measurement.

The coating film was dried at a temperature condition of 200° C. for 1 hour. As a result, the conductive adhesion preventing film 1B of this example was formed.

After the film formation, the film thickness of the conductive adhesion preventing film 1B was measured as a step between the masked non-film-formed part and the film surface of the conductive adhesion preventing film 1B and found to be 5 μm. For measuring the film thickness, a nano search microscope OLS 4500 (trade name; manufactured by Olympus Corporation) was used.

### Examples 2 to 6

In Examples 2 to 6, the material and the film thickness of Example 1 and the base material 4 are common, and the material, the length, the diameter, and the content ratio of the linear conductor 5 are changed. Hereinafter, differences from the first embodiment will be mainly described.

Example 2 is different from Example 1 in that a nickel (Ni) wire having a length of 200 μm and a diameter of 200 nm is used as the linear conductor 5.

Example 3 is different from Example 1 in that a silver (Ag) wire having a length of 40 μm and a diameter of 50 nm is used as the linear conductor 5.

Example 4 is different from Example 3 in that the diameter of the silver wire is changed to 70 nm.

Example 5 is different from Example 4 in that the amount of silver wire is changed to 5% by mass.

Example 6 is different from Example 4 in that the amount of the silver wire is changed to 40% by mass.

### Examples 7, 8

Examples 7 and 8 are examples in which the material of the base material 4 is changed in Example 4. Hereinafter, differences from the fourth embodiment will be mainly described.

Example 7 is different from Example 4 in that a fluororesin (abbreviated as “fluorine” in Table 1) is used as the base material 4. As the fluororesin, Teflon (registered trademark) PTFE dispersion 31-JR (trade name; manufactured by Du Pont-Mitsui Fluorochemicals Co., Ltd.) was used.

Example 8 is different from Example 4 in that silica is used as the base material 4. As the base material 4, Seracoat 22 (trade name; manufactured by Audec Corporation) which is a ceramic coating agent containing silica as a main component was used.

### Examples 9 to 16

Examples 9 to 16 are examples of the conductive adhesion preventing film 21B of the first modification. In Examples 9 to 16, conductive particles 25 were added to the paint used for forming the conductive adhesion preventing film 1B of Example 4 and manufactured. However, the amount of the linear conductor 5 in the paint was adjusted such that the amount of the linear conductor 5 in each of the conductive adhesion preventing films 21B was 10% by mass similar to that in Example 4. As the material of the conductive particles 25, copper particles were used.

In Example 9, copper particles having a particle diameter of 0.1 μm were contained by 5% by mass as the conductive particles 25. As the copper particles, a spherical copper powder Culox 6100 (trade name; manufactured by Culox) were used.

Hereinafter, with respect to Examples 10 to 16, differences from Example 9 will be mainly described.

Example 10 is different from Example 9 in that copper particles having a particle size of 0.5 μm were contained as the conductive particles 25. Wet copper powder Cu 1030 Y (trade name; manufactured by Mitsui Mining & Smelting Co., Ltd.) was used as the copper particles.

Example 11 is different from Example 9 in that copper particles having a particle diameter of 5.5 μm were contained as the conductive particles 25. Wet copper powder Cu 1400 Y (trade name; manufactured by Mitsui Mining & Smelting Co., Ltd.) was used as the copper particles.

Example 12 is different from Example 9 in that copper particles having a particle diameter of 8.2 μm were contained as the conductive particles 25. As the copper particles, fine atomized copper powder MA-CO8J (trade name; manufactured by Mitsui Mining & Smelting Co., Ltd.) was used.

Example 13 is different from Example 9 in that copper particles having a particle diameter of 10.7 μm were contained as the conductive particles 25. As copper particles, atomized copper powder Cu-HWQ 10 μm (trade name; manufactured by Fukuda Metal Foil Powder Industry Co., Ltd.) was used.

Example 14 is different from Example 11 in that the amount of copper particles was changed to 1% by mass.

Example 15 is different from Example 11 in that the amount of copper particles was changed to 3% by mass.

Example 16 is different from Example 11 in that the amount of copper particles was changed to 10% by mass.

### Comparative Examples 1 to 6

With respect to Comparative Examples 1 to 6, differences from the above Examples will be mainly described.

Comparative Example 1 is different from Example 1 in that a linear conductor having a length of 5 μm and a diameter of 150 nm was used instead of the linear conductor 5 of Example 1.

Comparative Example 2 is different from Example 3 in that a linear conductor having a length of 35 μm and a diameter of 40 nm was used instead of the linear conductor 5 of Example 3.

Comparative Example 3 is different from Example 4 in that the amount of the linear conductor 5 in Example 4 was changed to 3% by mass.

Comparative Example 4 is different from Example 4 in that the amount of the linear conductor 5 of Example 4 was changed to 50% by mass.

Comparative Example 5 is different from Example 16 in that the amount of the linear conductor 5 of Example 16 was changed to 0% by mass, and the linear conductor was not contained.

Comparative Example 6 is different from that of Example 16 in that the amount of the linear conductor 5 in Example 16 was changed to 0% by mass, the linear conductor was not contained, and the amount of the copper particle was changed to 50% by mass.

### Evaluation Method

Adhesion prevention evaluation, conductivity evaluation, and durability evaluation were performed on the test samples of Examples 1 to 16 and Comparative Examples 1 to 6.

In the adhesion prevention evaluation, the sample to be tested was heated to 200° C. on a hot plate, and the horse's blood was dripped as a biomaterial thereon. The sample to be tested was taken out of the hot plate 10 seconds after the horse's blood was drooped and cooled to room temperature. Thereafter, a tape peeling test by the cross cut method based on JIS K 5600-5-6 was performed on the sample to be tested.

In the test sample after the test, the peeled state of the solidified material of the blood of the horse was visually evaluated by the evaluator. The peeled state was classified based on the classification of Table 1 described in JIS K 5600-5-6 and evaluated as in the “adhesion prevention property” column of Table 1.

When the peeled state corresponds to “classification 0 to 4”, it was evaluated as “adhering” (described as “x” (no good) in Table 1).

When the peeled-off state corresponds to “classification 5”, the evaluator magnified and observed by using an optical microscope (DSX-500, manufactured by Olympus Co., Ltd.), and it was evaluated as either “no adhesion at all” (described as “@” (very good) in Table 1) or “slightly adhered” (described as “◯” (good) in Table 1).

In each of the test samples of Examples 1 to 16 and Comparative Examples 1 to 6, part or all of the adhesion preventing film for medical device did not peel off together with the solidified product of the blood of the horse.

In the conductivity evaluation, the volume resistivity of the sample to be tested was measured.

When the volume resistivity is 1.0×10⁶ Ω·cm or less, the conductivity was evaluated as “good” (described as “◯” (good) in Table 1). When the volume resistivity exceeds 1.0×10⁶ Ω·cm, the conductivity was evaluated as “no good” (described as “x” (no good) in Table 1).

In the durability evaluation, a scratch test of the test sample was performed, and the volume resistivity of the sample to be tested was measured before and after the marring test. In the scratch test, using a HEIDON Surface Property Measuring Machine (manufactured by Shinto Scientific Co., Ltd.), a test sample was subjected to 100 reciprocations in a state where a load of 0.98 N was applied with a flat indenter of 30×20 mm.

When the increase in the volume resistivity is equal to or less than 10% after the marring test compared to before the scratch test, the durability was evaluated as “good” (described as “◯” (good) in Table 1). When the increase in the volume resistivity exceeds 10%, the durability was evaluated as “no good” (described as “x” (no good) in Table 1).

The overall evaluation was evaluated in three stages, “very good” (described as “⊚” (very good) in Table 1), “good” (described as “◯” (good) in Table 1), and “no good” (described as “x” (no good) in Table 1).

“Very good” is the case where the conductivity evaluation and the durability evaluation are evaluated as “◯” (good) and the adhesion prevention evaluation is evaluated as “⊚” (very good).

“Good” is a case where adhesion prevention property evaluation, conductivity evaluation, and durability evaluation are evaluated as “◯” (good).

“No good” is a case where at least one of adhesion prevention property evaluation, conductivity evaluation, and durability evaluation is evaluated as “x” (no good).

### Evaluation Results

As shown in Table 1, in Examples 1, 2, 4 to 8, the overall evaluation was “good”. In Example 3, since the durability evaluation was “no good”, the overall evaluation was “no good”.

In Examples 9 to 16, the comprehensive evaluation of Examples 9 and 14 was “good”, and the other overall evaluations were “very good”.

It was considered that the reason why the adhesion preventing property evaluation in Example 9 remained “slightly adhered” was that the particle diameter of the copper particles was as small as 0.1 μm, so that the adhesion preventing property was not improved so much.

It was considered that the reason why the adhesion preventing property evaluation of Example 14 remained “slightly attached” was that the particle diameter of the copper particles was as small as 1% by mass even though it was the same as in Example 11, so it did not improve.

In Examples 10 to 13, 15 and 16, since the particle diameter of the copper particles was appropriate and the content ratio was appropriate, it was considered that adhesion prevention properties were improved as compared with Examples 1 to 8 not containing the conductive particles.

In contrast, the comprehensive evaluations of Comparative Examples 1 to 6 were all “no good”.

In Comparative Example 1, since the length of the linear conductor was 5 μm and was less than 10 μm, it is considered that the conductivity was lowered.

In Comparative Example 2, since the diameter of the linear conductor was 40 nm, which was less than 50 nm, the strength of the linear conductor was low. Therefore, it is considered that the durability became “no good” due to the breakage of the linear conductor in the marring test.

In Comparative Example 3, since the amount of the linear conductor was 3% by mass and was less than 5% by mass, it is considered that the conductivity was lowered.

In Comparative Example 4, since the amount of the linear conductor is 50% by mass and exceeds 40% by mass, it is considered that the adhesion preventing property has decreased.

Comparative examples 5 and 6 correspond to the above-described second comparative example (see FIG. 5). In either case, the copper particles are in contact with the electrode main body surface 1a and protrude to the outside of the base material 4 by a height of 0.5 μm, so that they have conductivity. However, in Comparative Example 5, since the amount of copper particles was too low, the volume resistivity is considered to be high.

In Comparative Example 6, since the amount of copper particles was high, the conductivity was “good”. However, the spacing between the exposed portions of the copper particles becomes too narrow, so adhesion prevention performance is considered to be deteriorated.

While preferred embodiments and modifications of the present invention have been described above in connection with the respective embodiments, the present invention is not limited to these embodiments, each modification, and each embodiment. Additions, omissions, substitutions, and other changes in the configuration are possible without departing from the spirit of the present invention.

Further, the present invention is not limited by the above description, and is limited only by the scope of the appended claims.

What is claimed is:

1. A conductive adhesion preventing film for medical use, comprising:
a nonconductive base material; and
a linear conductor having a length of 10 μm or more and a diameter of more than 50 nm and contained in the conductive adhesion preventing film by an amount of 5% by mass or more and 40% by mass or less,
wherein the conductive adhesion preventing film is formed on an electrode surface of a medical device performing at least one of incision, resection, coagulation, and ablation on living tissue by applying a high frequency voltage.

2. The conductive adhesion preventing film for medical use according to claim 1, wherein the nonconductive base material includes at least one material selected from a silica-based material, a silicone-based material, and a fluorine-based material.

3. The conductive adhesion preventing film for medical use according to claim 1, wherein the linear conductor has a length of 10 μm or more and 200 μm or less and a diameter of more than 50 nm and 200 nm or less.

4. The conductive adhesion preventing film for medical use according to claim 1, further comprising conductive particles having a particle diameter of 15 m or less, wherein
the linear conductor is contained in the conductive adhesion preventing film by 5% by mass or more and 30% by mass or less, and
the conductive particles is contained in the conductive adhesion preventing film by 10% by mass or less.

5. The conductive adhesion preventing film for medical use according to claim 4, wherein
a particle size of the conductive particles is 0.5 μm or more and 15 μm or less, and
the conductive particles is contained in the conductive adhesion preventing film by 3% by mass or more and 10% by mass or less.

6. The conductive adhesion preventing film for medical use according to claim 4, wherein a surface of the conductive particles is made of any one of silver, nickel, copper, and gold.

7. The conductive adhesion preventing film for medical use according to claim 4, wherein
the conductive particles comprise:
a particle body made of a nonconductive substance; and
a metal layer laminated on a surface of the particle body.

8. A medical device comprising a conductive adhesion preventing film for medical use, wherein
the conductive adhesion preventing film includes:
a nonconductive base material; and
a linear conductor having a length of 10 μm or more and a diameter of more than 50 nm and contained in the conductive adhesion preventing film by an amount of 5% by mass or more and 40% by mass or less, and
the conductive adhesion preventing film is formed on an electrode surface of a medical device performing at least one of incision, resection, coagulation, and ablation on living tissue by applying a high frequency voltage.
